(19) United States
(12) Patent Application Publication
(10) Pub. No.: US 2023/0274553 A1
(43) Pub. Date: Aug. 31, 2023

(54) IMAGE PROCESSING APPARATUS, IMAGE PROCESSING METHOD, AND NON-TRANSITORY STORAGE MEDIUM

(71) Applicant: NEC Corporation, Minato-ku, Tokyo (JP)

(72) Inventors: Gaku Nakano, Tokyo (JP); Shoji Nishimura, Tokyo (JP); Yoshitaka Higeta, Tokyo (JP); Youtarou Noda, Tokyo (JP); Shigeki Shinoda, Tokyo (JP); Yuzo Senda, Tokyo (JP); Shin Tominaga, Tokyo (JP); Masumi Ishikawa, Tokyo (JP); Daisuke Ikefuji, Tokyo (JP); Kana Takizawa, Tokyo (JP); Kenichi Ishii, Tokyo (JP); Yoshiyuki Satou, Tokyo (JP); Eishi Arita, Tokyo (JP); Shota Yamamoto, Tokyo (JP); Shinya Yamasaki, Tokyo (JP); Narumitsu Notomi, Tokyo (JP); Aki Nakamura, Tokyo (JP)

(73) Assignee: NEC Corporation, Minato-ku, Tokyo (JP)

(21) Appl. No.: 18/008,051

(22) PCT Filed: Mar. 8, 2021
(86) PCT No.: PCT/JP2021/008884

§ 371 Date: Dec. 2, 2022

(30) Foreign Application Priority Data
Jun. 5, 2020 (JP) 2020-098404

(51) Int. Cl. G06V 20/52; G06V 40/10

(52) U.S. Cl. CPC G06V 20/52; G06V 40/10

(57) ABSTRACT

The present invention provides an image processing apparatus (10) that includes: a feature value extraction unit (11) that, by processing an image including a plurality of persons, extracts an external appearance feature value of each of the persons; and a determination unit (12) that, by processing the image, determines another of the persons who satisfies an infection condition between him/her and each of the persons, and registers relevant information relating to the determined another person in association with the external appearance feature value of each of the persons.

### TECHNICAL FIELD

The present invention relates to an image processing apparatus, an image processing method, and a program.

### BACKGROUND ART

In recent years, image processing has been used for various purposes. For example, Patent Document 1 describes that, in a system that a droplet reaching range from a first target person is deviated from a respiration area of a second target person by adjusting an environment within a space, a position and an orientation of a face of each of the first target person and the second target person are determined by image processing.

### RELATED DOCUMENT

### Patent Document

[Patent Document 1] International Publication No. WO 2020/044826

### DISCLOSURE OF THE INVENTION

### Technical Problem

In order to prevent spread of an infectious disease, it is effective to determine a person who satisfies an infection condition between him/her and a person with an infectious disease, and test and isolate the determined person. The infection condition is a condition relating to possibility of infection, and does not necessarily mean that a person who satisfies the condition is always infected.

One of objects of the present invention is to enable determining a person who satisfies an infection condition between him/her and a predetermined person.

### Solution to Problem

According to the present invention, provided is an image processing apparatus including:
a feature value extraction unit that, by processing an image including a plurality of persons, extracts an external appearance feature value of each of the persons; and
a determination unit that, by processing the image, determines another of the persons who satisfies an infection condition between him/her and each of the persons, and registers relevant information relating to the determined another person in association with the external appearance feature value of each of the persons.

Further, according to the present invention, provided is an image processing method including,
by a computer:
extracting an external appearance feature value of each of the persons by processing an image including a plurality of persons; and
determining another of the persons who satisfies an infection condition between him/her and each of the persons, and registering relevant information relating to the determined another person in association with the external appearance feature value of each of the persons by processing the image.

Further, according to the present invention, provided is a program causing a computer to function as:
a feature value extraction unit that, by processing an image including a plurality of persons, extracts an external appearance feature value of each of the persons; and
a determination unit that, by processing the image, determines another of the persons who satisfies an infection condition between him/her and each of the persons, and registers relevant information relating to the determined another person in association with the external appearance feature value of each of the persons.

### Advantageous Effects of Invention

According to the present invention, a person who satisfies an infection condition between him/her and a predetermined person can be determined.

### BRIEF DESCRIPTION OF THE DRAWINGS

FIG. 1 is one example of a function block diagram of an image processing apparatus according to a present example embodiment.

FIG. 2 is a diagram for describing a usage environment of the image processing apparatus according to the present example embodiment.

FIG. 3 is a diagram schematically illustrating one example of information processed by the image processing apparatus according to the present example embodiment.

FIG. 4 is a diagram schematically illustrating one example of information processed by the image processing apparatus according to the present example embodiment.

FIG. 5 is a diagram for describing one example of a processing content of the image processing apparatus according to the present example embodiment.

FIG. 6 is a flowchart illustrating one example of a processing flow of the image processing apparatus according to the present example embodiment.

FIG. 7 is a diagram illustrating one example of a hardware configuration of the image processing apparatus according to the present example embodiment.

FIG. 8 is one example of a function block diagram of the image processing apparatus according to the present example embodiment.

FIG. 9 is a flowchart illustrating one example of a processing flow of the image processing apparatus according to the present example embodiment.

FIG. 10 is one example of an image output by the image processing apparatus according to the present example embodiment.

FIG. 11 is one example of an image output by the image processing apparatus according to the present example embodiment.

### DESCRIPTION OF EMBODIMENTS

Hereinafter, example embodiments of the present invention will be described by using the drawings. Note that, a similar component is assigned with a similar reference sign throughout all the drawings, and description therefor will be omitted as appropriate.

### First Example Embodiment

### “Overview”

An image processing apparatus according to a present example embodiment includes a function of processing an image including a plurality of persons, thereby determining a combination of persons who mutually satisfy an infection condition from among the persons included in the image, and generating a database indicating a determination result. Use of the database enables determining a person who satisfies an infection condition between him/her and a predetermined person (for example, a person with an infectious disease). Hereinafter, detailed description will be given.

### “Configuration”

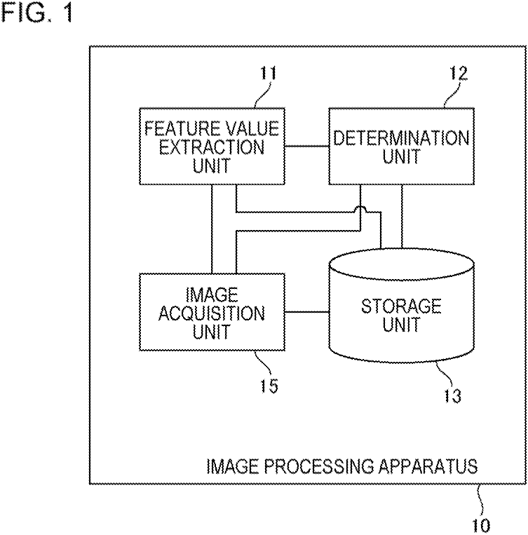

Next, a function configuration of the image processing apparatus will be described. FIG. 1 illustrates one example of a function block diagram of an image processing apparatus 10. As illustrated, the image processing apparatus 10 includes a feature value extraction unit 11, a determination unit 12, a storage unit 13, and an image acquisition unit 15. Note that, the image processing apparatus 10 may not include the storage unit 13. In this case, an external apparatus being configured communicably with the image processing apparatus 10 includes the storage unit 13.

The image acquisition unit 15 acquires an image including a plurality of persons generated by an image capturing apparatus. The image acquisition unit 15 can store the acquired image in the storage unit 13. When the image processing apparatus 10 and the image capturing apparatus are communicably connected to each other, the image acquisition unit 15 can receive an image from the image capturing apparatus. Besides the above, an image generated by the image capturing apparatus may be stored in any storage apparatus. Then, the image acquisition unit 15 may acquire an image stored in the storage apparatus at any timing. The image acquisition unit 15 may acquire the image generated by the image capturing apparatus by batch processing, or may acquire the image by real-time processing.

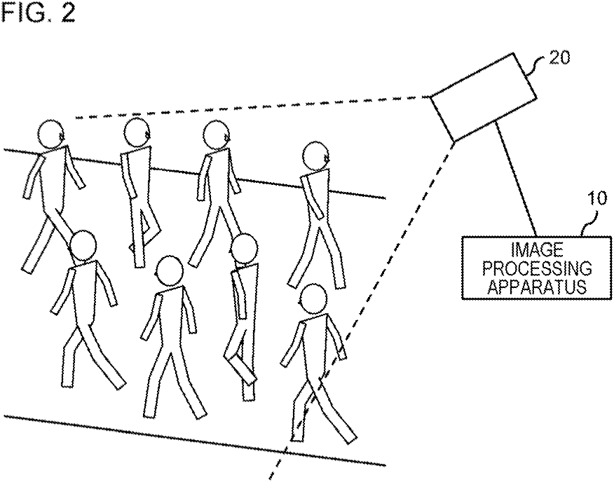

Herein, the image capturing apparatus will be described. FIG. 2 is a diagram for describing a usage environment of the image processing apparatus 10 according to the example embodiment. The image processing apparatus 10 is used together with an image capturing apparatus 20.

The image capturing apparatus 20 is, for example, a fixed camera, and repeatedly photographs a region (hereinafter, described as a target region) where a plurality of persons, for example, an unspecified large number of persons come and go. Thus, an image generated by the image capturing apparatus 20 includes a plurality of persons. The image capturing apparatus 20 may be, for example, a surveillance camera installed in a store, an institution, a facility, or the like. An image generated by the image capturing apparatus 20 has any frame rate, but may have, for example, a frame rate that constitutes a moving image. As one example, the image capturing apparatus 20 can be communicably connected to the image processing apparatus 10, and transmit the generated image to the image processing apparatus 10.

In an example illustrated in FIG. 2, the image processing apparatus 10 is connected to one image capturing apparatus 20. However, the image processing apparatus 10 may be connected to a plurality of image capturing apparatuses 20. In this case, the plurality of image capturing apparatuses 20 capture mutually different target regions. Further, each of the plurality of image capturing apparatuses 20 transmits, to outside, an image in association with information (hereinafter, described as image capturing apparatus identification information) identifying the image capturing apparatus 20. By doing so, a desired analysis can be performed for each of a plurality of, for example, a large number of target regions at hundred places or more.

Returning to FIG. 1, the feature value extraction unit 11 processes an image including the plurality of persons acquired by the image acquisition unit 15, and thereby detects a person from the image. Then, the image acquisition unit 15 extracts an external appearance feature value (face information or the like) of each of the detected persons. Note that, the feature value extraction unit 11 may estimate, based on the image, an attribute of each of the detected persons. The attribute is capable of being estimated from the image, and is, for example, “classification of adult, child, and elder”, “age”, “gender”, and the like. Detection of a person, extraction of a feature value, and estimation of an attribute are widely known techniques, and thus, description herein is omitted.

The feature value extraction unit 11 can determine an identical person who is present redundantly in different images by using, for example, the external appearance feature value or location information of the detected person. In addition, the feature value extraction unit 11 can identify the plurality of detected persons from one another by using, for example, the external appearance feature value.

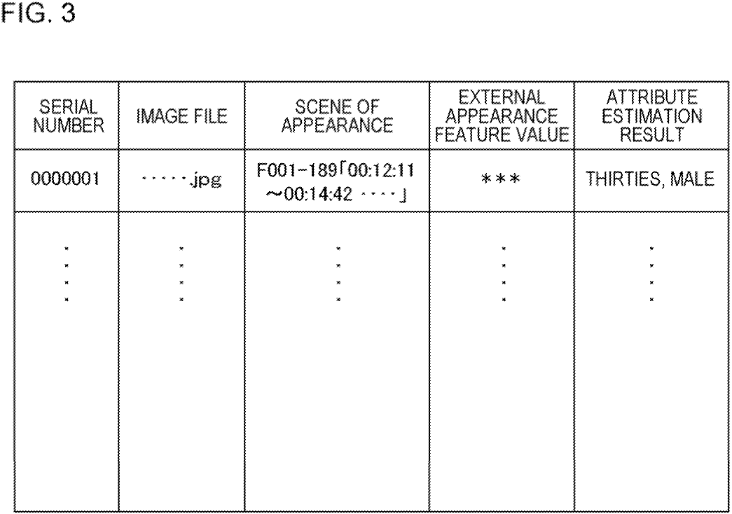

Through the above processing by the feature value extraction unit 11, for example, information as illustrated in FIG. 3 is generated. The information is stored in the storage unit 13. The information illustrated in FIG. 3 is information on a person detected from the image, and includes a “serial number”, an “image file”, a “scene of appearance”, an “external appearance feature value”, and an “attribute estimation result”. Note that, some of the items may not be included, or other items may be included.

The “serial number” is identification information assigned to each of the detected persons. One serial number is assigned to one person.

The “image file” indicates a file of a representative image among a plurality of images including each person. For example, an image in which each person is captured largest, an image in which a face of each person is detected, or the like is a representative image.

The “scene of appearance” is information indicating a scene in which each person appears. For example, a scene of appearance (a detected scene) may be explicitly indicated by a file name of a moving image and a lapse time or the like from beginning. Besides the above, a scene in which each of the detected persons appears may be explicitly indicated by a file name of an image in which each person is detected.

The “external appearance feature value” indicates an external appearance feature value of each person extracted by the feature value extraction unit 11.

The “attribute estimation result” is a result of estimating an attribute of each person estimated by the feature value extraction unit 11. For example, “classification of adult, child, and elder”, “age”, “gender”, and the like are included.

Returning to FIG. 1, the determination unit 12 processes an image including the plurality of persons acquired by the image acquisition unit 15, and thereby determines another of the persons who satisfies an infection condition between him/her and each of the detected persons. Then, the determination unit 12 registers relevant information relating to the determined another person in association with an external appearance feature value (information identifying each person) of each of the detected persons.

Herein, the infection condition will be described. The infection condition is a condition relating to possibility of infection, and does not necessarily mean that a person who satisfies the condition is always infected. The infection condition is defined, for example, based on at least one of a mutual distance, an orientation of a face of a person, a wearing article of a person, a movement of a mouth of a person, and a physical contact.

For example, the infection condition may be that one or a plurality of following conditions are satisfied. Besides the above, the infection condition may be that a duration of a state where one or a plurality of the following conditions are satisfied satisfies a reference value or more. Besides the above, a frequency with which a state where one or a plurality of the following conditions are satisfied occurs (the number of times, a proportion of time that the state is satisfied to a predetermined period of time, or the like) may be equal to or more than a reference value.

(Condition 1) A mutual distance is equal to or less than a reference value.

(Condition 2) At least one is directing an orientation of his/her face toward another one.

(Condition 3) At least one is not wearing a predetermined wearing article on his/her face.

(Condition 4) At least one is moving his/her mouth.

(Condition 5) A physical contact has been made with each other.

(Condition 6) A predetermined pattern of physical contact has been made with each other. The predetermined pattern is “holding hands”, “handshake”, “hug”, “kiss”, or the like.

The reference value in the condition 1 is determined based on a so-called social distance. The social distance is a physical distance to be kept between adjacent persons in order to prevent infection of an infectious disease. In addition, magnitude of the reference value is set based on a main route of infection of a target infectious disease. For example, regarding an infectious disease caused mainly by droplet infection, a value being equal to or more than 1.5 m and equal to or less than 6 m is used for the reference value. Further, regarding an infectious disease caused mainly by contact infection, a value being equal to or more than 50 cm and equal to or less than 1.5 m is used for the reference value. Note that, hereinafter, one example of a method of computing a distance between two persons based on an image will be described.

The wearing article in the condition 3 is for covering at least one (preferably both) of a mouth and a nose, and is, for example, a mask or a muffler.

Detecting a movement of a mouth in the condition 4 enables detecting an operation such as talking, laughing, or sneezing.

Besides the above, the infection condition may be “touching the same object”. The object is an object that is located within a photographing region of the image capturing apparatus 20 and is fixed at that place (that is, an object that is always present within a capturing region of the image capturing apparatus 20), and examples thereof include, for example, but not limited to, a strap, a vending machine, a ticket machine, a self-accounting apparatus, and the like. In a case of the examples, a plurality of persons who have not been present at the same place at the same timing may mutually satisfy the infection condition.

Next, the relevant information will be described. The relevant information includes at least one of an image of the determined another person, an external appearance feature value of the determined another person, a result of estimating an attribute of the determined another person, and a content of the infection condition satisfied between each person and the determined another person.

The content of the infection condition satisfied between each person and the determined another person may be information indicating what condition is satisfied. This assumes a case where there are a plurality of conditions. Besides the above, the content of the infection condition satisfied between each person and the determined another person may further include detailed information thereof. For example, the content of the infection condition satisfied between each person and the determined another person may include “a shortest mutual distance”, “a duration for which a mutual distance is equal to or less than a reference value”, “a frequency with which a mutual distance is equal to or less than a reference value”, “whether one or both are directing an orientation of his/her face toward another one”, “whether one or both are not wearing a predetermined wearing article on his/her face”, “whether one or both are moving his/her mouth”, “a duration of physical contact with each other”, “a frequency of physical contact with each other”, “what pattern of contact has been made”, and the like.

Further, the relevant information may include an infection risk being computed based on the content of the infection condition satisfied between each person and the determined another person. The infection risk is computed by the determination unit 12. An algorithm for computing the infection risk is not particularly limited, but is designed in such a way as to satisfy a following content.

For example, when the above-described condition 1 is satisfied, the infection risk increases as a shortest mutual distance decreases. Further, when the above-described condition 2 is satisfied, the infection risk increases when both are directing an orientation of his/her face toward another one, more than when one is directing an orientation of his/her face toward another one. Further, when the above-described condition 3 is satisfied, the infection risk increases when both are not wearing a predetermined wearing article on his/her face, more than when one is not wearing a predetermined wearing article on his/her face.

Further, when the above-described condition 4 is satisfied, the infection risk increases when both are moving his/her mouth, more than when one is moving his/her mouth. Further, when the above-described condition 5 or 6 is satisfied, the infection risk increases as a pattern of contact more susceptible to infection is made. In this case, susceptibility to infection for each of a plurality of patterns of contact is registered in the processing apparatus 10.

Further, when any of the above-described conditions 1 to 6 is satisfied, the infection risk increases as a duration of a state where the condition is satisfied becomes longer. Further, when any of the above-described conditions 1 to 6 is satisfied, the infection risk increases as a frequency with which a state where the condition is satisfied occurs (the number of times, a proportion of time that the state is satisfied to a predetermined period of time, or the like) becomes higher.

Besides the above, the relevant information may include information indicating at least one of a timing and a place at which the infection condition is satisfied. The timing at which the infection condition is satisfied may be indicated by date and time information (year-month-day and time), or may be indicated by a lapse time or the like from beginning of a moving image file. The information indicating a place may be identification information of the image capturing apparatus 20 that has photographed the scene, may be an address of the place, may be a name of the place, or may be latitude and longitude information. Note that, when an address, a name, latitude and longitude information, or the like of the place is employed, an address, a name, latitude and longitude information, or the like of an installation place of each image capturing apparatus 20 is registered in advance in the image processing apparatus 10, in association with each image capturing apparatus 20.

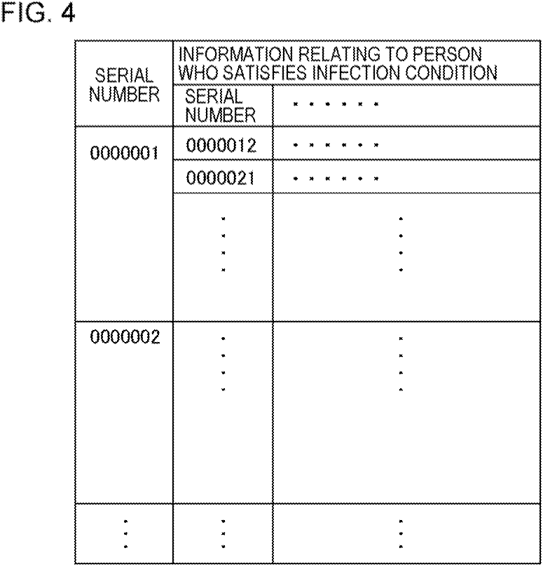

FIG. 4 schematically illustrates one example of information generated by the determination unit 12. The information is stored in the storage unit 13. In an illustrated example, the relevant information (in the figure, “information relating to a person who satisfies an infection condition”) relating to the determined another person is registered in association with a serial number of each person. The relevant information includes the serial number and the like. The serial number is identification information assigned to each of the detected persons, as described above.

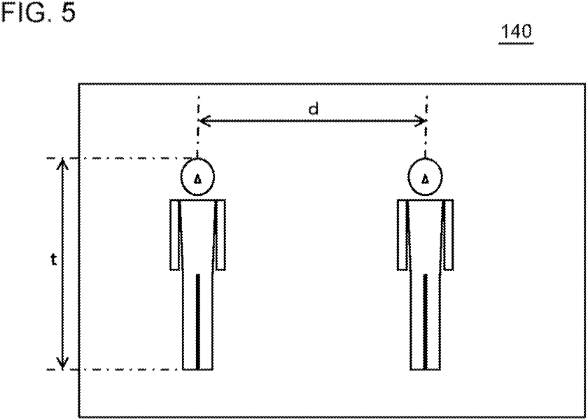

Next, one example of a method of computing a distance between two persons, based on an image, will be described by using FIG. 5. First, the determination unit 12 computes a height t of a reference person or another person in an image. Herein, t is represented by, for example, the number of pixels. Then, the determination unit 12 computes a distance d from the reference person to the another person in the image. Herein, d is represented by the same unit (for example, the number of pixels) as t. Then, the determination unit 12 computes d/t, multiplies the value d/t by the above-described reference body height, and thereby computes a distance between the reference person and the another person.

Note that, the reference body height is set in advance. The reference body height may be changed depending on a place (for example, a country) where the image capturing apparatus 20 is installed. For example, as the reference body height, an average body height of adults in a country where the target image capturing apparatus 20 is installed is used. As an example of specific processing, the image processing apparatus 10 stores information determining the reference body height for each image capturing apparatus 20. Then, the determination unit 12 reads out and uses the reference body height related to the image capturing apparatus 20 that has generated an image to be processed.

Further, when an attribute (for example, at least one of gender and an age group) of a person the height t of which is to be computed can be estimated by image processing, the determination unit 12 may change the reference body height depending on the attribute.

Note that, most of images have a distortion specific to the image capturing apparatus 20 that has generated the image. When computing a distance between two persons, the determination unit 12 preferably performs processing of correcting the distortion. The determination unit 12 performs distortion correction processing according to a position of a person in an image. In general, the distortion of an image results from, for example, an optical system (for example, a lens) included in the image capturing apparatus 20 and a vertical orientation (for example, an angle with respect to a horizontal plane) of the image capturing apparatus 20. In view of this, a content of the distortion correction processing according to a position of a person in an image is set according to an optical system (for example, a lens) included in the image capturing apparatus 20 and a vertical orientation of the image capturing apparatus 20.

Note that, in the processing described by using the present figure, when an object having a size standardized to some extent is included in an image, the determination unit 12 may compute a distance between two persons by using a size of the object instead of a body height of a person.

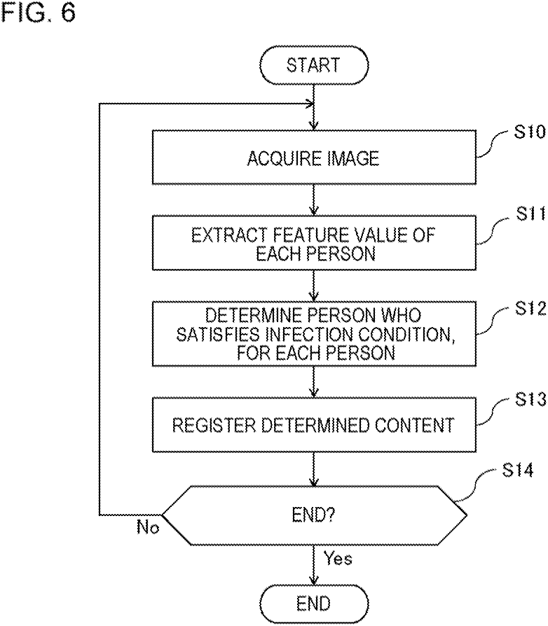

Next, one example of a processing flow of the image processing apparatus 10 will be described by using a flowchart in FIG. 6.

When the image acquisition unit 15 acquires one image (S10), the feature value extraction unit 11 detects a person from the image. Then, the feature value extraction unit 11 extracts an external appearance feature value of the detected person, and updates information (see FIG. 3) stored in the storage unit 13 (S11). Thereafter, the determination unit 12 determines a person who satisfies an infection condition for each person detected from the image (S12), and updates information (see FIG. 4) stored in the storage unit 13 (S13). Thereafter, the image processing apparatus 10 returns to S10 and repeats similar processing.

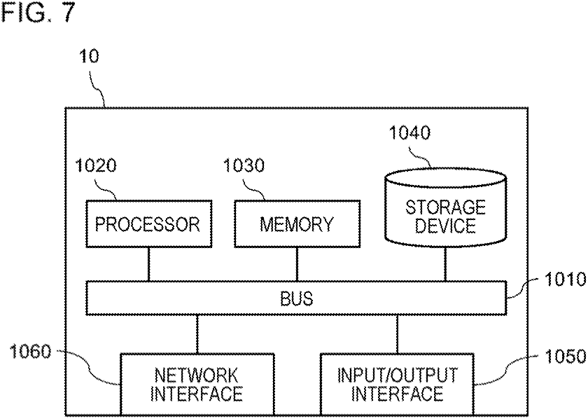

Next, one example of a hardware configuration of the image processing apparatus 10 will be described. FIG. 7 is a diagram illustrating a hardware configuration example of the image processing apparatus 10. The image processing apparatus 10 includes a bus 1010, a processor 1020, a memory 1030, a storage device 1040, an input/output interface 1050, and a network interface 1060.

The bus 1010 is a data transmission path through which the processor 1020, the memory 1030, the storage device 1040, the input/output interface 1050, and the network interface 1060 transmit and receive data to and from one another. However, a method of connecting the processor 1020 and the like with one another is not limited to bus connection.

The processor 1020 is a processor achieved by a central processing unit (CPU), a graphics processing unit (GPU), or the like.

The memory 1030 is a main storage apparatus achieved by a random access memory (RAM) or the like.

The storage device 1040 is an auxiliary storage apparatus achieved by a hard disk drive (HDD), a solid state drive (SSD), a memory card, a read only memory (ROM), or the like. The storage device 1040 stores a program module for achieving each function of the image processing apparatus 10. Each of the program modules is read into the memory 1030 and executed by the processor 1020, and thereby each function relevant to the program module is achieved Further, the storage device 1040 also functions as the storage unit 13.

The input/output interface 1050 is an interface for connecting the image processing apparatus 10 to various types of input/output equipment with each other.

The network interface 1060 is an interface for connecting the image processing apparatus 10 to a network. The network is, for example, a local area network (LAN) or a wide area network (WAN). A method by which the network interface 1060 connects to the network may be wireless connection, or may be wired connection. The image processing apparatus 10 may communicate with the image capturing apparatus 20 via the network interface 1060.

### “Advantageous Effect”

The image processing apparatus 10 according to the present example embodiment can process an image including a plurality of persons, thereby determine a combination of persons who mutually satisfy an infection condition from among the persons included in the image, and generate a database indicating a determination result. Use of the database enables determining a person who satisfies an infection condition between him/her and a predetermined person (for example, a person with an infectious disease).

Further, the image processing apparatus 10 can register, as information relevant to a person who satisfies an infection condition between him/her and each person, an image of the person, an external appearance feature value of the person, and a result of estimating an attribute of the person. Use of the pieces of information enables finding a person who satisfies an infection condition between him/her and a predetermined person (for example, a person with an infectious disease).

Further, the image processing apparatus 10 can register, as information relevant to a person who satisfies an infection condition between him/her and each person, a result of estimating an attribute of the person. Use of the information enables further searching (narrowing down), from among persons who satisfy an infection condition between him/her and a predetermined person (for example, a person with an infectious disease), for a person who pertains to a predetermined attribute.

Further, the image processing apparatus 10 can register, as information relevant to a person who satisfies an infection condition between him/her and each person, a content of the infection condition satisfied between him/her and each person. Use of the information enables further searching (narrowing down), from among persons who satisfy an infection condition between him/her and a predetermined person (for example, a person with an infectious disease), for a person whose content of the infection condition satisfied between him/her and the predetermined person satisfies a predetermined condition.

Further, the image processing apparatus 10 can register, as information relevant to a person who satisfies an infection condition between him/her and each person, an infection risk being computed based on a content of the infection condition satisfied between him/her and each person. Use of the information enables further searching (narrowing down), from among persons who satisfy an infection condition between him/her and a predetermined person (for example, a person with an infectious disease), for a person whose infection risk satisfies a predetermined condition.

Further, the image processing apparatus 10 can detect a person who satisfies an infection condition being defined based on at least one of a mutual distance, an orientation of a face of a person, a wearing article of a person, a movement of a mouth of a person, and a physical contact. Further, the image processing apparatus 10 can detect a person who satisfies an infection condition being “touching the same object”. The image processing apparatus 10 as described above can accurately detect a person who has possibility of infection from each person.

### Second Example Embodiment

An image processing apparatus 10 according to a present example embodiment is different from the image processing apparatus 10 according to the first example embodiment in a point of further including a function of searching for desired information from a database being generated by the function described in the first example embodiment.

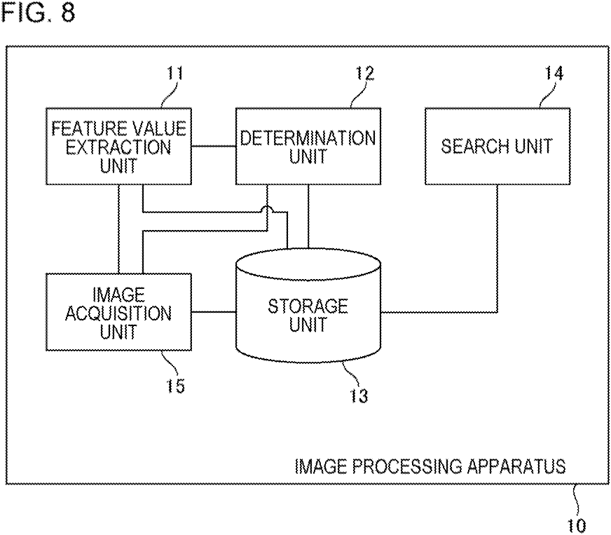

FIG. 8 illustrates one example of a function block diagram of the image processing apparatus 10 according to the present example embodiment. The image processing apparatus 10 according to the present example embodiment is different from the image processing apparatus 10 according to the first example embodiment in a point of including a search unit 14.

The search unit 14 searches for and outputs relevant information associated with a specified external appearance feature value. For example, an image including a person may be input as a search condition. In this case, the search unit 14 analyzes the image and extracts an external appearance feature value of a person included in the image. Then, the search unit 14 searches for relevant information stored in a storage unit 13, by using the extracted external appearance feature value as a search query.

Note that, a search condition may be defined by using other information included in relevant information. For example, a search condition may be defined by using information included in “a content of an infection condition satisfied between each person and a determined another person” described in the first example embodiment. For example, desired relevant information may be searched for by using “what condition is satisfied”, “a shortest mutual distance”, “a duration for which a mutual distance is equal to or less than a reference value”, “a frequency with which a mutual distance is equal to or less than a reference value”, “whether one or both are directing an orientation of his/her face toward another one”, “whether one or both are not wearing a predetermined wearing article on his/her face”, “whether one or both are moving his/her mouth”, “a duration of physical contact with each other”, “a frequency of physical contact with each other”, “what pattern of contact has been made”, “an infection risk”, “a place at which the infection condition is satisfied”, “a timing at which the infection condition is satisfied”, and the like.

The search unit 14 outputs a search result (relevant information matching a search condition) via a predetermined output apparatus. The output apparatus is, but not limited to, a display, a projection apparatus, and the like.

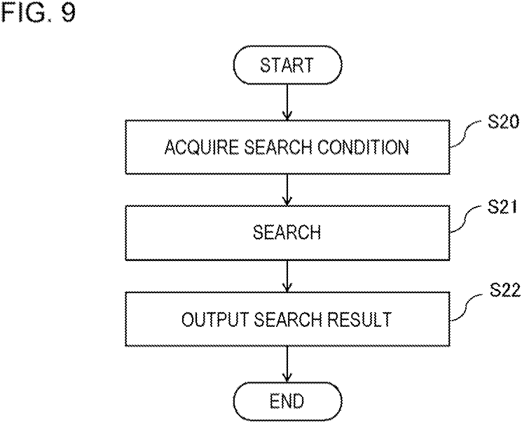

Next, one example of a processing flow of the image processing apparatus 10 will be described by using a flowchart in FIG. 9. When the search unit 14 acquires a search condition (S20), the search unit 14 searches for relevant information matching the search condition (S21), and outputs a search result (S22).

Other configurations of the image processing apparatus 10 according to the present example embodiment are similar to the configurations of the image processing apparatus 10 according to the first example embodiment.

The image processing apparatus 10 according to the present example embodiment can achieve an advantageous effect similar to the image processing apparatus 10 according to the first example embodiment. Further, the image processing apparatus 10 according to the present example embodiment can search for a desired person, for example, a person who satisfies an infection condition between him/her and a predetermined person (for example, a person with an infectious disease), a person who satisfies an infection condition between him/her and a predetermined person in a predetermined mode, and the like. Further, the predetermined mode can be specified by various types of items such as “what condition is satisfied”, “a shortest mutual distance”, “a duration for which a mutual distance is equal to or less than a reference value”, “a frequency with which a mutual distance is equal to or less than a reference value”, “whether one or both are directing an orientation of his/her face toward another one”, “whether one or both are not wearing a predetermined wearing article on his/her face”, “whether one or both are moving his/her mouth”, “a duration of physical contact with each other”, “a frequency of physical contact with each other”, “what pattern of contact has been made”, “an infection risk”, “a place at which the infection condition is satisfied”, “a timing at which the infection condition is satisfied”, and the like. Thus, desired relevant information can be efficiently searched for.

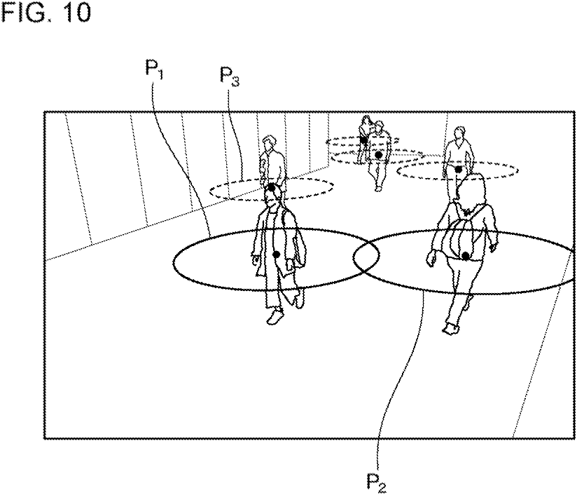

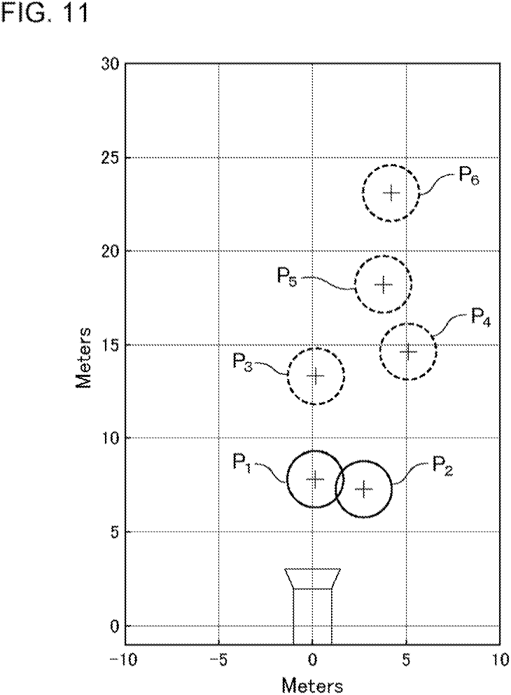

Herein, a modification example will be described. The image processing apparatus 10 may display an image as illustrated in FIGS. 10 and 11 on an output apparatus such as a display. In the figures, the image processing apparatus 10 displays, for each person, a mark that indicates a range of a recommendation value (for example, the above-described reference value) for a social distance centering on the person. Then, when a mark related to a certain person overlaps a mark related to a nearby person, that is, when a distance between a certain person and a nearby person becomes equal to or less than the recommendation value for the social distance, the image processing apparatus 10 displays the marks (for example, persons P₁ and P₂ in FIGS. 10 and 11) related to each of the two persons in a mode different from the mark (for example, persons P₃, P₄, P₅, and P₆ in FIGS. 10 and 11) related to another person. Examples of a way of making a mode different include, for example, changing a display color, and making a mode of a line (a solid line, a dotted line, a dashed line, and the like) forming the mark different. In a case of changing a display color, for example, the image processing apparatus 10 displays the mark of a normal state in blue and, when two marks overlap each other, displays the two marks in red.

In an example illustrated in FIG. 10, the image processing apparatus 10 superimposes the above-described mark on an image (a moving image in some cases) being used when generating infection risk information. On the other hand, in an example illustrated in FIG. 11, the image processing apparatus 10 superimposes the above-described mark on a plan view by which arrangement of persons is indicated. The image processing apparatus 10 may display the display illustrated in FIG. 10 and the display illustrated in FIG. 11 simultaneously on an output apparatus.

While the example embodiments of the present invention have been described with reference to the drawings, the example embodiments are illustrative of the present invention, and various configurations other than the above can be employed.

Further, while a plurality of processes (pieces of processing) are described in order in a plurality of flowcharts used in the above description, execution order of processes executed in each example embodiment is not limited to the described order. The order of the illustrated processes can be changed in each example embodiment, as long as the change does not detract from contents. Further, each of the above example embodiments can be combined, as long as contents do not contradict each other.

The whole or part of the above-described example embodiments can be described as, but not limited to, the following supplementary notes.

1. An image processing apparatus including:
a feature value extraction unit that, by processing an image including a plurality of persons, extracts an external appearance feature value of each of the persons; and
a determination unit that, by processing the image, determines another of the persons who satisfies an infection condition between him/her and each of the persons, and registers relevant information relating to the determined another person in association with the external appearance feature value of each of the persons.

2. The image processing apparatus according to supplementary note 1, further including

a search unit that searches for and outputs the relevant information associated with the specified external appearance feature value.

3. The image processing apparatus according to supplementary note 1 or 2, wherein

The relevant information includes at least one of an image of the another person, the external appearance feature value of the another person, a result of estimating an attribute of the another person, and a content of the infection condition satisfied between him/her and each of the persons.

4. The image processing apparatus according to any one of supplementary notes 1 to 3, wherein

The relevant information includes an infection risk being computed based on a content of the infection condition satisfied between him/her and each of the persons.

5. The image processing apparatus according to any one of supplementary notes 1 to 4, wherein

The relevant information includes information indicating at least one of a timing and a place at which the infection condition is satisfied.

6. The image processing apparatus according to any one of supplementary notes 1 to 5, wherein

The infection condition is defined based on at least one of a mutual distance, an orientation of a face of the person, a wearing article of the person, a movement of a mouth of the person, and a physical contact.

7. The image processing apparatus according to any one of supplementary notes 1 to 6, wherein

The infection condition is touching a same object as the person has touched.

8. An image processing method including,
by a computer:
extracting an external appearance feature value of each of the persons by processing an image including a plurality of persons; and
determining another of the persons who satisfies an infection condition between him/her and each of the persons, and registering relevant information relating to the determined another person in association with the external appearance feature value of each of the persons by processing the image,.

9. A program causing a computer to function as:
a feature value extraction unit that, by processing an image including a plurality of persons, extracts an external appearance feature value of each of the persons; and
a determination unit that, by processing the image, determines another of the persons who satisfies an infection condition between him/her and each of the persons, and registers relevant information relating to the determined another person in association with the external appearance feature value of each of the persons.

This application is based upon and claims the benefit of priority from Japanese patent application No. 2020-098404, filed on Jun. 5, 2020, the disclosure of which is incorporated herein in its entirety by reference.

Reference signs List

| | |
|---|---|
| 10 | Image processing apparatus |
| 11 | Feature value extraction unit |
| 12 | Determination unit |
| 13 | Storage unit |
| 14 | Search unit |
| 15 | Image acquisition unit |

What is claimed is:

1. An image processing apparatus comprising:
at least one memory configured to store one or more instructions; and
at least one processor configured to execute the one or more instructions to:
extract, by processing an image including a plurality of persons, an external appearance feature value of each of the persons; and
determine another of the persons who satisfies an infection condition between him/her and each of the persons, and register relevant information relating to the determined another person in association with the external appearance feature value of each of the persons by processing the image.

2. The image processing apparatus according to claim 1,
wherein the processor is further configured to execute the one or more instructions to search for and output the relevant information associated with the specified external appearance feature value.

3. The image processing apparatus according to claim 1, wherein
the relevant information includes at least one of an image of the another person, the external appearance feature value of the another person, a result of estimating an attribute of the another person, and a content of the infection condition satisfied between him/her and each of the persons.

4. The image processing apparatus according to claim 1, wherein
the relevant information includes an infection risk being computed based on a content of the infection condition satisfied between him/her and each of the persons.

5. The image processing apparatus according to claim 1, wherein
the relevant information includes information indicating at least one of a timing and a place at which the infection condition is satisfied.

6. The image processing apparatus according to claim 1, wherein
the infection condition is defined based on at least one of a mutual distance, an orientation of a face of the person, a wearing article of the person, a movement of a mouth of the person, and a physical contact.

7. The image processing apparatus according to claim 1, wherein
the infection condition is touching a same object as the person has touched.

8. An image processing method comprising,
by a computer:
extracting an external appearance feature value of each of the persons by processing an image including a plurality of persons; and
determining another of the persons who satisfies an infection condition between him/her and each of the persons, and registering relevant information relating to the determined another person in association with the external appearance feature value of each of the persons by processing the image.

9. A non-transitory storage medium storing a program causing a computer to :
extract, by processing an image including a plurality of persons, an external appearance feature value of each of the persons; and
determine another of the persons who satisfies an infection condition between him/her and each of the persons, and register relevant information relating to the determined another person in association with the external appearance feature value of each of the persons by processing the image.
